(19) United States
(12) Patent Application Publication
(10) Pub. No.: US 2011/0245231 A1
(43) Pub. Date: Oct. 6, 2011

(54) ACTIVE SUBSTANCE AND PHARMACEUTICAL COMPOSITION FOR TREATING ALCOHOL DEPENDENCE, AND A METHOD FOR OBTAINING AND THE USE OF SAID ACTIVE SUBSTANCE

(75) Inventors: Andrey Alexandrovich Ivashchenko, Moscow (RU); Nikolay Filippovich Savchuk, Rancho Santa Fe, CA (US)

(73) Assignee: Alla Chem, LLC, Carson City, NV (US)

(21) Appl. No.: 13/133,948

(22) PCT Filed: Dec. 15, 2009
(86) PCT No.: PCT/RU09/00691

§ 371 Date: Jun. 10, 2011

(51) Int. Cl. A61K 31/554; C07D 401/12; A61K 31/4439; C07D 235/28; A61K 31/4184; C07D 405/12; C07D 409/12; C07D 413/12; A61K 31/4245; A61K 31/5377; A61K 31/454; C07D 403/12; A61K 31/4192; A61K 31/496; A61K 31/4985; A61P 25/24; A61P 25/32

(52) U.S. Cl. 514/211.13; 546/273.7; 514/338; 548/307.1; 514/395; 548/304.7; 548/131; 514/364; 544/139; 514/234.5; 546/199; 514/322; 548/255; 514/359; 544/370; 514/254.06; 514/250

(57) ABSTRACT

The invention relates to novel drug substance for the treatment of alcohol dependence, pharmaceutical composition, medicament and method for treatment of dependence on using ethyl alcohol containing beverages.

The invention provides a drug substance for treating alcohol dependence in human and warm-blooded animals representing substituted 1H-benzimidazoles of the general formula 1 or pharmaceutically acceptable salts and/or hydrates thereof

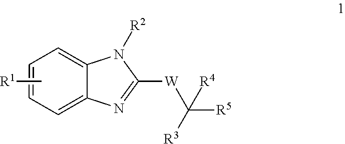

wherein: W represents S or S═0 group; R¹ represents one or more substituent selected from hydrogen, halogen, C₁-C₄ alkyl, C₁-C₄ alkoxy, optionally substituted azaheterocyclyl; R² represents hydrogen or optionally substituted C₁-C₄ alkyl; R³ and R⁴ independently represent optionally identical substituents selected from hydrogen or optionally substituted C₁-C₄ alkyl; R⁵ represents an alkyl substituent selected from hydrogen, optionally substituted C₁-C₇ alkyl, optionally substituted aryl, optionally substituted heterocyclyl, C₁-C₄ alkoxycarbonyl, optionally substituted aminocarbonyl.

### FIELD OF THE INVENTION

The invention is directed to the drug substance for treatment of alcohol dependence, pharmaceutical composition, medicament and method for treatment of alcohol dependence induced by excessive intake of alcohol containing beverages.

### BACKGROUND OF THE INVENTION

Most of the people indulging products which contain ethyl alcohol in the period of abstention from the use of them suffer from formidable attraction (“desire”) to alcohol. This alcohol dependence results in repeating periods of excessive use of alcohol comprising products.

By now a great number of medicaments (drugs) for treatment of alcohol dependence have been offered, the most known of which are, for example, Naltrexone, Acamprosate and sodium γ-hydroxybutyrate (SHB).

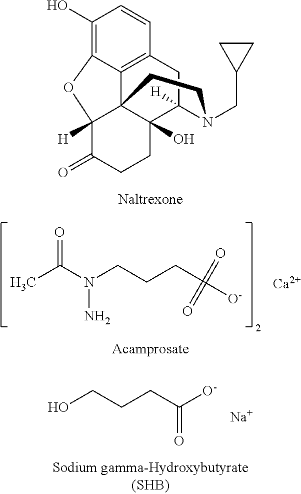

Naltrexone, falling into the category of opiate receptor antagonists, taken together with alcohol, ameliorates alcoholic dependence that results in reduction of indulged amount of alcohol [Pat. RU 2090190]. It is known, however, that the main contraindication limiting the utility of Naltrexone is liver insufficiency [Krystal J. H., Cramer J. A., Krol W. F., Kirk G. F., Rosenheck R. A.; Veterans Affairs Naltrexone Cooperative Study 425 Group. Naltrexone in the treatment of alcohol dependence. N. Engl. J. Med. 2001, 345(24):1734-9].

Acamprosate also suppresses alcohol dependence, which manifests itself in moderate lowering of alcohol intake in the future [Moncrieff J., Drummond D. C. New drug treatments for alcohol problems: a critical appraisal. Addiction. 1997, vol. 92, pp. 939-47; discussion, see pp. 949-64]. However, liver insufficiency is also the main contraindication limiting the usage of Acamprosate [Williams S. H. Medications for treating alcohol dependence. Am. Fam. Physician. 2005, 72(9):1775-80].

A medicament comprising as active ingredients γ-hydroxybutyric acid or its salts is known, which being taken during the period of abstinence reduces alcohol dependence, the result of which is decreasing the number of repeated relapses of excessive drinking in the future [Nava F., Premi S., Manzato E, Campagnola W, Lucchini A, Gessa G. L. Gamma-hydroxybutyrate reduces both withdrawal syndrome and hypercortisolism in severe abstinent alcoholics: an open study vs. diazepam. Am. J. Drug Alcohol Abuse. 2007, 33: 379-392; 2007]. It is also known that regular usage of γ-hydroxybutyric acid itself or its salts may cause addiction (pharmacomania) that hinders its safe use at alcohol abusers [Sumnall H. R., Woolfall K., Edwards S., Cole J. C., Beynon C. M. Use, function, and subjective experiences of gamma-hydroxybutyrate (GHB), Drug Alcohol Depend. 2008, 92(1-3):286-90], That is why, γ-hydroxybutyric acid or its salts are used mainly as reference substances in pre-clinical study.

Searching for effective and safe remedies for alcoholism treatment stays on to be an important problem of modern medicine. There are known drug candidates for alcohol dependence treatment, which are at the stage of clinical trial now. Thus, for example, in 2006 Varenicline tartrate has appeared in the market [EP 1078637, EP 1159970, EP 1177798], Neramexane hydrochloride is in the III phase of clinical trial [US 2006002999, U.S. Pat. No. 6,071,966], the drug substances MTIP [WO 2006102194] and CVT-10216 [WO 2008014497] are at the stage of pre-clinical investigation

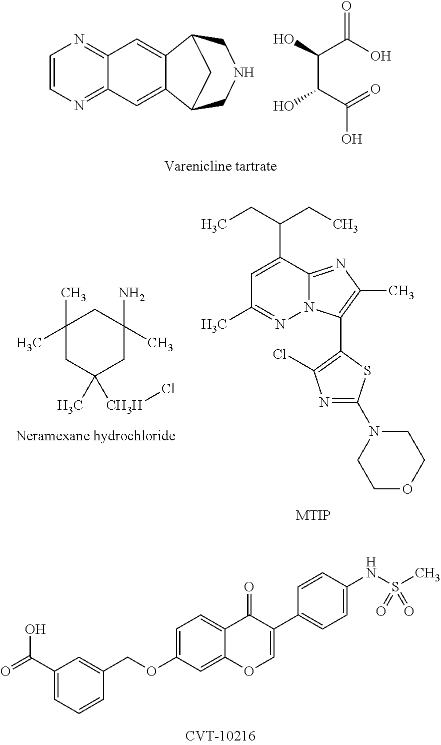

Substituted 1H-benzimidazoles of the general formula 1 and pharmaceutically acceptable salts and/or hydrates thereof exhibiting various types of pharmacological activity are known

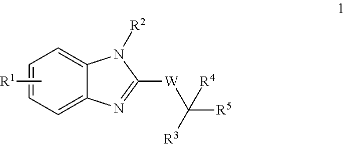

wherein:
W represents S or S═O group;
R¹ represents one or more substituents selected from hydrogen, halogen, C₁-C₄ alkyl, C₁-C₄ alkyloxy, optionally substituted azaheterocyclyl;
R² represents hydrogen or optionally substituted C₁-C₄ alkyl;
R³ and R⁴ independently of each other represent optionally identical substituents, selected from hydrogen or optionally substituted C₁-C₄ alkyl;
R⁵ represents an alkyl substituent selected from hydrogen, optionally substituted C₁-C₇ alkyl, optionally substituted aryl, optionally substituted heterocyclyl, C₁-C₄ alkoxycarbonyl, optionally substituted aminocarbonyl.

Table 1 presents known substituted 1H-benzimidazoles of the general formula 1 and their pharmacological activity.

TABLE 1
Pharmacologically active 1H-benzimidazoles of the general formula 1.

| No | | Therapeutic | |
|---|---|---|---|
| compound | Formula | indications | Patent |
| 1.1 | 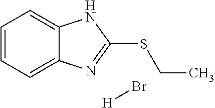 | Nootropic, memory enhancement, liver protection | SU 1251374; RU 2188012 |
| 1.2 | 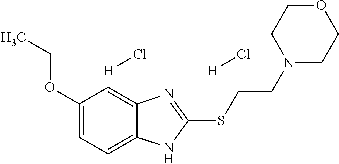 | Anxiolytic, treatment of anxious and cognitive disorders | EP 0788795 |
| 1.3 | 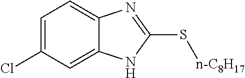 | Treatment of gastric ulcer and duodenal ulcer | US 5106863 |
| 1.4 | 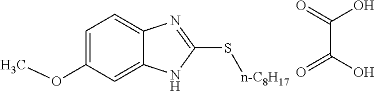 | Treatment of gastric ulcer and duodenal ulcer | US 5106863 |
| 1.5 | 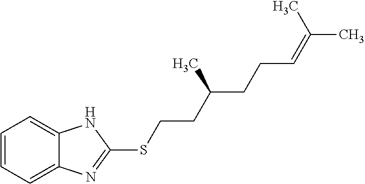 | Treatment of gastric ulcer and duodenal ulcer | US 5106863 |
| 1.6 | 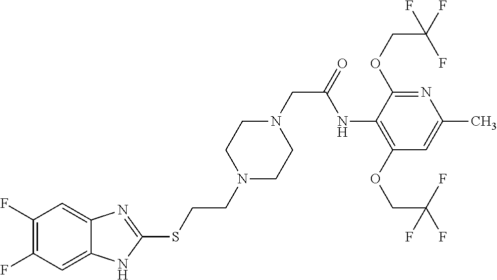 | Treatment of lipoprotein disorders | WO 2005003119 |
| 1.7 | 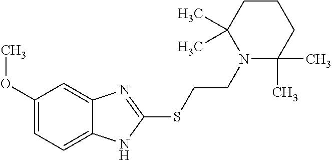 | Treatment of gastric ulcer and duodenal ulcer | EP 0457331 |
| 1.8 | 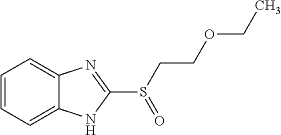 | Treatment of gastric ulcer and duodenal ulcer | EP 0370436 |
| 1.9 | 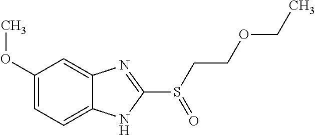 | Treatment of gastric ulcer and duodenal ulcer | EP 0452076 |
| 1.10 | 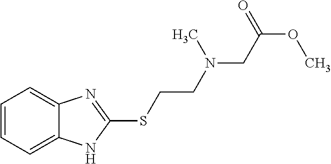 | Treatment of gastric ulcer and duodenal ulcer | JP 1991014566 |
| 1.11 | 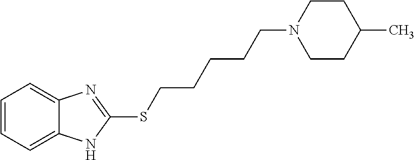 | Treatment of atherosclerosis | WO 2001000588 |
| 1.12 | 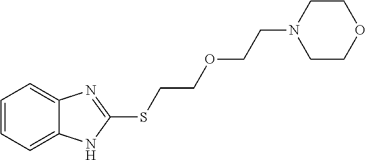 | Treatment of atherosclerosis | WO 2001000588 |
| 1.13 | 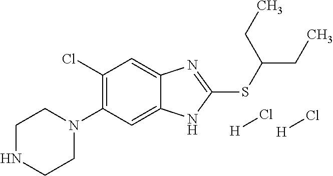 | Analgesic, treatment of pains | WO 2002040019 |
| 1.14 | 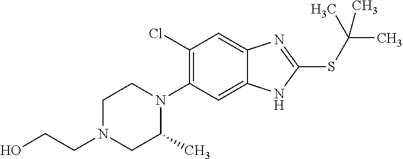 | Analgesic, treatment of pains | WO 2002040019 |
| 1.15 | 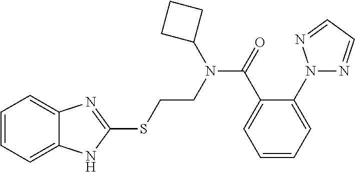 | Treatment of obesity and sleep disorders | WO 2007126934 |
| 1.16 | 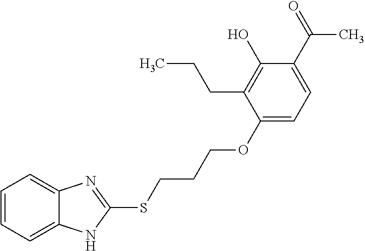 | Treatment of asthma and allergy | EP 0287971 (B1) |
| 1.17 | 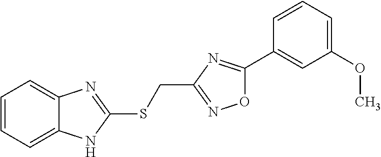 | Anxiolytic, analgesics, treatment of anxious and cognitive disorders, treatment of pains | WO 2004014881; WO 20050773465 |

Other commercially available ChemDiv Inc. [www.chemdiv.com] substituted 1H-benzimidazoles of the general formula 1 are known, some of them are presented in Table 2.

TABLE 2
Commercially available 1H-benzimidazoles of the general formula 1.

| No | | No | |
|---|---|---|---|
| com- | | com- | |
| pound | Formula | pound | FormulaΦopmyπa |
| 1.18 | 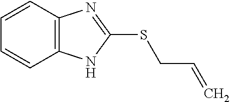 | 1.36 | 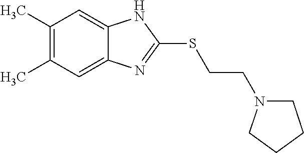 |
| 1.19 | 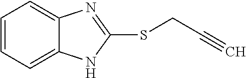 | 1.37 | 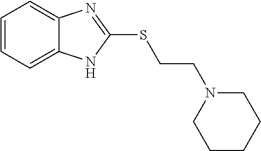 |
| 1.20 | 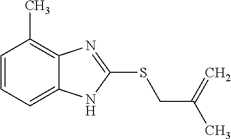 | 1.38 | 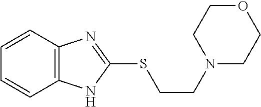 |
| 1.21 | 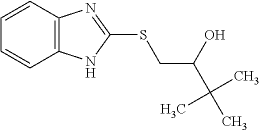 | 1.39 | 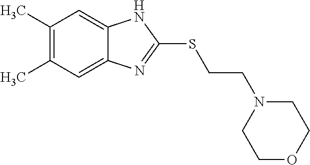 |
| 1.22 | 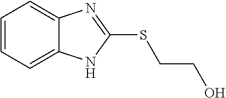 | 1.40 | 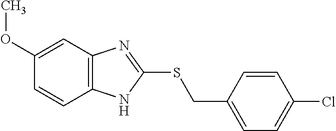 |
| 1.23 | 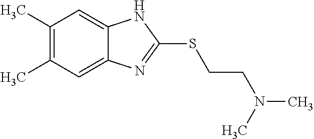 | 1.41 | 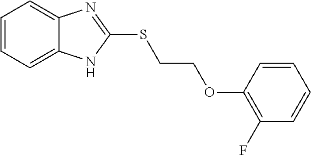 |
| 1.24 | 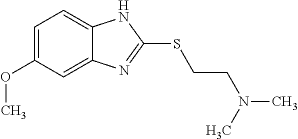 | 1.42 | 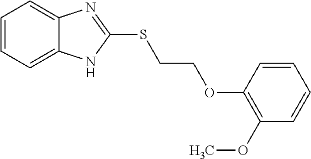 |
| 1.25 | 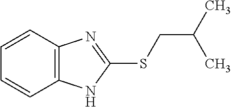 | 1.43 | 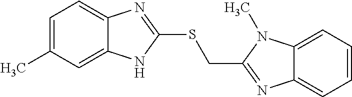 |
| 1.26 | 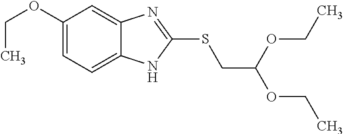 | 1.44 | 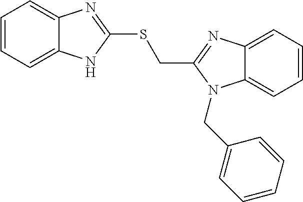 |
| 1.27 | 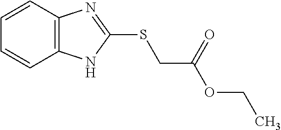 | 1.45 | 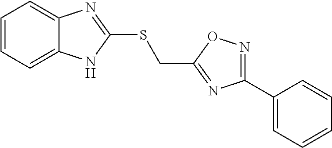 |
| 1.28 | 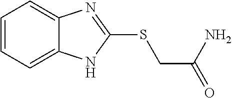 | 1.46 | 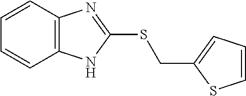 |
| 1.29 | 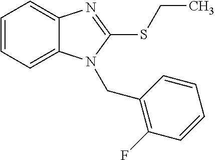 | 1.47 | 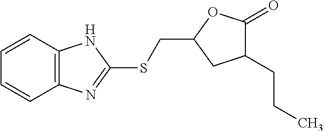 |
| 1.30 | 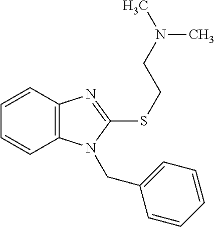 | 1.48 | 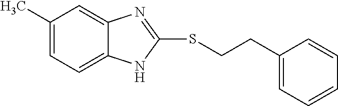 |
| 1.31 | 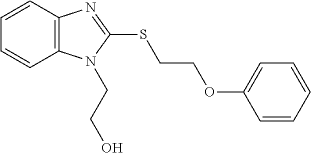 | 1.49 | 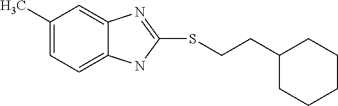 |
| 1.32 | 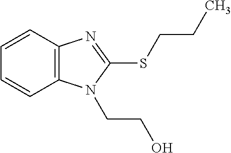 | 1.50 | 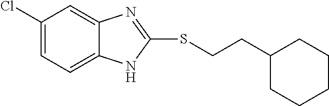 |
| 1.33 | 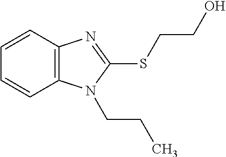 | 1.51 | 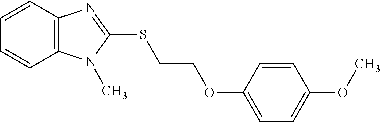 |
| 1.34 | 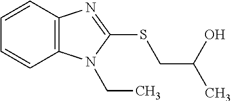 | 1.52 | 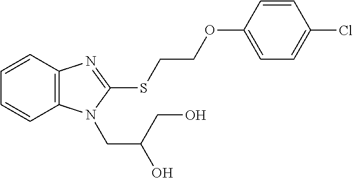 |
| 1.35 | 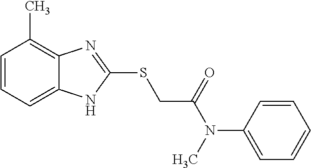 | 1.53 | 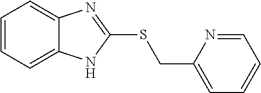 |

A drug substance 2-ethylsulfanyl-1H-benzimidazole hydrobromide (Bemithyl) 1.1 with nootropic and antiasthenic action is known [SU 1251374], it is also effective in improving the processes of liver regeneration [RU 2188012], in particular, it has a protective influence on alcohol abusers' liver [Okovityai S. B., Ivanova O. B., Schabanov P. D. Bemithyl hepatoprotective effect at patients with long-lasting alcohol-induced liver injuries. Narcology, 2002, No 3, p. 19-23].

A drug substance for treatment of anxious disorders—2-[2-(morpholin-4-yl)-ethylsulfanyl]-5-ethyloxy-1H-benzimidazole dihydrochloride (Afobazole) 1.2, is known [EP 0788795].

However, either Bemithyl 1.1 or Afobazole 1.2, as well as other substituted 1H-benzimidazoles of the general formula 1 presented in Table 1 have never been used for treating of alcohol dependence.

### DISCLOSURE OF THE INVENTION

In the context of the invention, the following terms, unless otherwise indicated, shall be understood to have the following meanings:

“Azaheterocycle” means aromatic or non-aromatic mono- or polycyclic system comprising at least one nitrogen atom in the cycle. Azaheterocycle may have one or more “cyclic system substituents”.
“Alkyl” means aliphatic hydrocarbon straight or branched group with 1-12 carbon atoms. Branched means alkyl chain with one or more “lower alkyl” side substituents. Alkyl group may have one or more substituents of the same or different structure (“alkyl substituent”) including halogen, alkenyloxy, cycloalkyl, aryl, heteroaryl, heterocyclyl, aroyl, cyano, hydroxy, alkoxy, carboxy, alkynyloxy, aralkoxy, aryloxy, aryloxycarbonyl, alkylthio, heteroarylthio aralkylthio, arylsulfonyl, or Rₖ^{a}Rₖ₊₁^{a}N—, Rₖ^{a}Rₖ₊₁^{a}NC(═O)—, Rₖ^{a}Rₖ₊₁^{a}NC(═S)—, Rₖ^{a}Rₖ₊₁^{a}NSO₂—, where Rₖ^{a} and Rₖ₊₁^{a} independently of each other represent “amino group substituent”, the meanings of which are defined in this section, for example, hydrogen, alkyl, aryl, aralkyl, heteroaralkyl, heterocyclyl or heteroaryl, or Rₖ^{a} and Rₖ₊₁^{a} together with the nitrogen atom, they are attached to, form through Rₖ^{a} and Rₖ₊₁^{a} 4-7-membered heterocyclyl or heterocyclenyl. The preferred alkyl groups are methyl, trifluoromethyl, cyclopropylmethyl, cyclopentylmethyl, ethyl, n-propyl, iso-propyl, n-butyl, tert-butyl, n-pentyl, 3-pentyl, methoxyethyl, carboxymethyl, methoxycarbonylmethyl, ethoxycarbonylmethyl, benzyloxycarbonylmethyl and pyridylmethyloxycarbonylmethyl. The preferred “alkyl substituents” are cycloalkyl, aryl, heteroaryl, heterocyclyl, hydroxy, alkoxy, alkoxycarbonyl, aralkoxy, aryloxy, alkylthio, heteroarylthio, aralkylthio, alkylsulfonyl, arylsulfonyl, alkoxycarbonyl, aralkoxycarbonyl, heteroaralkyloxycarbonyl or Rₖ^{a}Rₖ₊₁^{a}N—, Rₖ^{a}Rₖ₊₁^{a}NC(═O)—, annelated arylheterocyclenyl, annelated arylheterocyclyl.
“Alkylamino” means CₙH₂ₙ₊₁NH— or (CₙH₂₊₁)(CₙH₂₊₁)N— groups, in which the meaning of alkyl is defined herein. The preferred alkylamino groups are methylamino, ethylamino, n-propylamino, iso-propylamino and n-butylamino.
“Alkyloxy” means CₙH₂ₙ₊₁O— group, in which alkyl is defined herein. The preferred alkyloxy groups are methyloxy, ethyloxy, n-propyloxy, iso-ptopyloxy and n-butyloxy.
“Alkyloxycarbonyl” means —C(O)OCₙH₂₊₁ group, in which alkyl is defined herein. The preferred alkyloxycabonyl groups are methoxycarbonyl, ethoxycarbonyl, n-butoxycarbonyl, iso-propyloxycarbonyl, tert.-butyloxycarbonyl, benzyloxycarbonyl and phenethyloxycarbonyl.
“Amino group” means Rₖ^{a}Rₖ₊₁^{a}N— group, substituted or unsubstituted with optionally identical “amino group substituents” Rₖ^{a} and Rₖ₊₁^{a}, the meanings of which are defined herein, for example, amino (H₂N—), methylamino, diethylamino, pyrrolidino, morpholino, benzylamino or phenethylamino.
“Aryl” means aromatic mono- or polycyclic system with 6-14 carbon atoms, predominantly from 6 to 10 C-atoms. Aryl may have one or more “cyclic system substituents” of the same or different structure. Phenyl, substituted phenyl, naphthyl, or substituted naphthyl are the representatives of aryl groups. Aryl could be annelated with nonaromatic cyclic system or heterocycle.
“Halogen” means fluorine, chlorine, bromine and iodine. Preference is given to fluorine, chlorine and bromine.
“Hydrate” means stoichiometric or nonstoichiometric compositions of the compounds or their salts with water.
“Heterocycle” means aromatic or non-aromatic mono- or poly-cyclic system comprising in the cycle at least one heteroatom. The preferred heteroatoms are N, O and S. Heterocycle may have one or more “cyclic system substituents”.
“Heterocyclyl” means a radical derived from heterocycle.
“Substituent” means a chemical radical attached to the scaffold (fragment), for example, “alkyl substituent”, “amino group substituent”, “carbamoyl substituent”, and “cyclic system substituent”, the meanings of which are defined herein.
“Cyclic system substituent” means a substituent attached to aromatic or non-aromatic cyclic system, including hydrogen, alkyl, alkenyl, alkynyl, aryl, heteroaryl, aralkyl, heteroaralkyl, hydroxy, hydroxyalkyl, amino, aminoalkyl, alkoxy, aryloxy, acyl, aroyl, halogen, nitro, cyano, carboxy, alkyloxycarbonyl, aryloxycarbonyl, aralkoxycarbonyl, alkyloxyalkyl, aryloxyalkyl, heterocyclyloxyalkyl, arylalkyloxyalkyl, heterocyclylalkyloxyalkyl, alkylsulfonyl, arylsulfonyl, heterocyclylsulfonyl, alkylsulfinyl, arylsulfinyl, heterocyclylsulfinyl, alkylthio, arylthio, heterocyclylthio, alkylsulfonylalkyl, arylsulfonylalkyl, heterocyclylsulfonylalkyl, alkylsylfinylalkyl, arylsulfinylalkyl, heterocyclylsulfinylalkyl, alkylthioalkyl, arylthioalkyl, heterocyclylthioalkyl, arylalkylsulfonylalkyl, heterocyclylalkylsulfonylalkyl, arylalkylthioalkyl, heterocyclylalkylthioalkyl, cycloalkyl, cycloalkenyl, heterocyclyl, heterocyclenyl, amidino, Rₖ^{a}Rₖ₊₁^{a}N—, Rₖ^{a}N═, Rₖ^{a}Rₖ₊₁^{a}N-alkyl-, Rₖ^{a}Rₖ₊₁^{a}NC(═O)— or Rₖ^{a}Rₖ₊₁^{a}NSO₂—, wherein Rₖ^{a} and Rₖ₊₁^{a} independently represent “amino group substituents”, the meanings of which are defined in this section, for example, hydrogen, optionally substituted alkyl, optionally substituted aryl, optionally substituted aralkyl or optionally substituted heteroaralkyl, or a substituent Rₖ^{a}Rₖ₊₁^{a}N— which Rₖ^{a} could be acyl or aroyl, the meaning of Rₖ₊₁^{a} is defined above, or “cyclic system substituents” are Rₖ^{a}Rₖ₊₁^{a}NC(═O)— or Rₖ^{a}Rₖ₊₁^{a}NSO₂—, in which Rₖ^{a} and Rₖ₊₁^{a} together with the nitrogen atom they are attached to form through Rₖ^{a} and Rₖ₊₁^{a} 4-7 membered heterocyclyl or heterocyclenyl.
“Drug substance” means physiologically active compound of synthetic or other (biotechnological, vegetable, animal, microbe and so on) origin exhibiting pharmacological activity and being an active ingredient of pharmaceutical composition employed in preparation and production of medicaments.
“Medicament”—is a compound or a mixture of compounds representing a pharmaceutical composition in the form of tablets, capsules, injections, ointments and other drug products intended for restoration, improvement or modification of physiological functions at humans and animals, and for prophylaxis and treatment of diseases, diagnostics, anesthesia, contraception, cosmetology and others.
“Lower alkyl” means straight or branched alkyl with 1-4 carbon atoms.
“Pharmaceutical composition” means composition comprising, at least, one of the compounds of the general formula 1 and, at least, one of the components selected from pharmaceutically acceptable and pharmacologically compatible fillers, solvents, diluents, auxiliaries, distributing and sensing agents, delivery agents, such as preservatives, stabilizers, disintegrators, moisteners, emulsifiers, suspending agents, thickeners, sweeteners, flavoring agents, aromatizing agents, antibacterial agents, fungicides, lubricants, and prolonged delivery controllers, the choice and suitable proportions of which depend on the nature and way of administration and dose. Examples of suitable suspending agents are: ethoxylated isostearyl alcohol, polyoxyethene, sorbitol and sorbitol ether, microcrystalline cellulose, aluminum metahydroxide, bentonite, agar-agar and tragacant and their mixtures as well. Protection against microorganism action can be provided by various antibacterial and antifungal agents, such as: parabens, chlorobutanol, sorbic acid, and similar compounds. Composition may also contain isotonic agents, such as: sugar, sodium chloride, and similar compounds. Prolonged effect of the composition may be achieved by the agents inhibiting absorption of the active ingredient, for example, aluminum monostearate and gelatin. Examples of suitable carriers, solvents, diluents and delivery agents include water, ethanol, polyalcohols and their mixtures, natural oils (such as olive oil) and injection-grade organic esters (such as ethyl oleate). Examples of fillers are: lactose, milk-sugar, sodium citrate, calcium carbonate, calcium phosphate and the like. Examples of disintegrators and distributors are: starch, alginic acid and its salts, and silicates. Examples of suitable lubricants are: magnesium stearate, sodium lauryl sulfate, talc and polyethylene glycol of high molecular weight. Pharmaceutical composition for peroral, sublingual, transdermal, intramuscular, intravenous, subcutaneous, local or rectal administration of active ingredient, alone or in combination with another active compound, may be administered to humans and animals in standard administration form as a mixture with traditional pharmaceutical carriers. Suitable standard administration forms include peroral forms such as tablets, gelatin capsules, pills, powders, granules, chewing-gums and peroral solutions or suspensions, for example, therapeutic kit; sublingual and transbuccal administration forms; aerosols; implants; local, transdermal, subcutaneous, intramuscular, intravenous, intranasal or intraocular forms and rectal administration forms. Pharmaceutical compositions could be prepared, as a rule, by use of conventional procedures consisting in mixing together an active compound and liquid or reduced to powder carrier.
“Pharmaceutically acceptable salt” refers to relatively non-toxic organic or inorganic acid addition salts and base addition salts of compounds of this invention. These salts can be prepared in situ during the final isolation, purification or synthesis of the compounds or prepared specially. In particular, acid addition salts can be prepared by separately reacting the purified compounds in its free base form with a suitable organic or inorganic acid. Examplary acid addition salts include: hydrochloride, hydrobromide, sulfate, bisulfate, phosphate, nitrate, acetate, oxalate, valeriate, oleate, palmitate, stearate, laurate, borate, benzoate, lactate, p-toluenesulfonate, citrate, maleates, fumarates, succinates, tartrates, mesylate, malonates, salicylates, propionate, ethane sulfonates, benzene sulfonates, sulfamates and the like (Detailed description of such salts properties is given in: Berge S. M., et al., “Pharmaceutical Salts” J. Pharm. Sci., 1977, 66: 1-19). Salts of the disclosed acids may be prepared by the reaction of purified acids with a suitable base; moreover, metal salts and amine salts may be synthesized too. Metal salts are salts of sodium, potassium, calcium, barium, zinc, magnesium, lithium and aluminum; sodium and potassium salts are being preferred. Suitable inorganic compounds from which metal salts can be prepared are: sodium hydroxide, carbonate, bicarbonate and hydride; potassium hydroxide, carbonate and bicarbonate, lithium hydroxide, calcium hydroxide, magnesium hydroxide, zinc hydroxide. Organic bases suitable for preparation of the disclosed acid salts are amines and amino acids the basicity of which is high enough to produce stable salts suitable for medicinal purposes (in particular, they are to have low toxicity). Such amines include ammonia, methylamine, dimethylamine, trimethylamine, ethylamine, diethylamine, triethylamine, benzylamine, dibenzylamine, dicyclohexylamine, piperazine, ethylpiperidine, tris(hydroxymethyl)aminomethane and the like. Besides, salts can be prepared using some tetraalkylammonium hydroxides, such as: holine, tetramethylammonium, tetraethylammonium, and the like. Amino acids may be selected from the main amino acids—lysine, ornithine and arginine.

One embodiment of the present invention is a drug substance for treating of alcoholic dependence at humans and warm-blooded animals, representing substituted 1H-benzimidazoles of the general formula 1 and pharmaceutically acceptable salts and/or hydrates thereof.

The preferred drug substances are 2-ethylsulfanyl-1H-benzimidazole hydrobromide of formula 1.1 or 2-[2-(morpholin-4-yl)-ethylsulfanyl]-5-ethyloxy-1H-benzimidazole dihydrochloride of formula 1.2.

Another embodiment of the present invention is a pharmaceutical composition for treatment of alcoholic dependence at humans and warm-blooded animals comprising an effective dosage of the drug substance representing at least one substituted 1H-benzimidazole of the general formula 1 or pharmaceutically acceptable salts and/or hydrates thereof.

The preferred pharmaceutical composition comprises as the drug substance 2-ethylsulfanyl-1H-benzimidazole hydrobromide of formula 1.1 or 2-[2-(morpholin-4-yl)-ethylsulfanyl]-5-ethyloxy-1H-benzimidazole dihydrochloride of formula 1.2.

Yet another embodiment of the present invention is also a pharmaceutical composition for treatment of alcohol dependence in humans and warm-blooded animals comprising an effective dosage of the drug substance representing at least one substituted 1H-benzimidazole of the general formula 1 or pharmaceutically acceptable salts and/or hydrate thereof and an anti-depressant.

There is a synergistic effect of substituted 1H-benzimidazole of the general formula 1 and anti-depressant that results in more effective treatment of alcohol dependence at lower doses of the drug substance.

As anti-depressants can be used, for example, 5-methyl-2,3,7,8,9,10-hexahydro-1H-pyrazino[3,2,1-jk]carbazole hydrochloride of formula 2.1 (Pirazidole) [GB 1340528; RU0276060; WO 206048242], 5-cyclohexyl-2,3,7,8,9,10-hexahydro-1H-pyrazino[3,2,1-jk]carbazole hydrochloride of formula 2.2 (Tetrindol) [Glushkov, R. G.; Mashkovsky, M. D.; Andrejeva, N. I. Tetrindole. Drugs Fut., 1997, 22(12), 1333; Mashkovsky, M. D., Glushkov, R. G.; Schvedov V. I., Andrejeva, N. I., Golovina S. M. Exp. Clin. Pharmmacol., 1993, 56(2), 3-6], N-methyl-3-phenyl-3-(4-(trifluoromethyl)phenoxy)propane-1-amine 2.3 hydrochloride (Prozak) [EP 0830864; U.S. Pat. No. 4,314,081; U.S. Pat. No. 6,927,223; WO 1992018005; WO 2007064586], 2-(diphenyl-methanesulfinyl)-acetamide 2.4 (Modaphinyl) [EP 0462004; EP 0547952; EP 0594507; US 2007065517; U.S. Pat. No. 4,177,290; WO 1995000132; WO 2006030278; WO 2006032146], methyl-(2,3,3-trimethyl-bicyclo[2.2.1]hept-2-yl)-amine hydrochloride 2.5 (Mecamylamine) [U.S. Pat. No. 2,831,027; U.S. Pat. No. 6,034,079; WO 1999015492; WO 2007075720], 7-{4-[4-(2,3-dichlorophenyl)-piperazin-1-yl]-butoxy}-3,4-dihydro-1H-quinolin-2-one 2.6 (Aripiprazole) [EP 0367141; US 2005245541; WO 2002060423; WO 2007118923], 2-[2-(4-dibenzo[b,f][1,4]thiazepin-11-ylpiperazin-1-yl)-ethoxy]-ethanol fumarate 2.7 (Quetiapine fumarate) [EP 0240228; JP 2005060286; U.S. Pat. No. 6,599,897; WO 1997045124; WO 2007058593], 1-(3-dimethylamino-propyl)-1-(4-fluorophenyl)-1,3-duhydro-isobenzofurane-5-carbonitrile hydrochloride 2.8 (Nitalapram)[CA 2163840; EP 0474580; US 2002061925; WO 2000012044; WO 2006103550] and others.

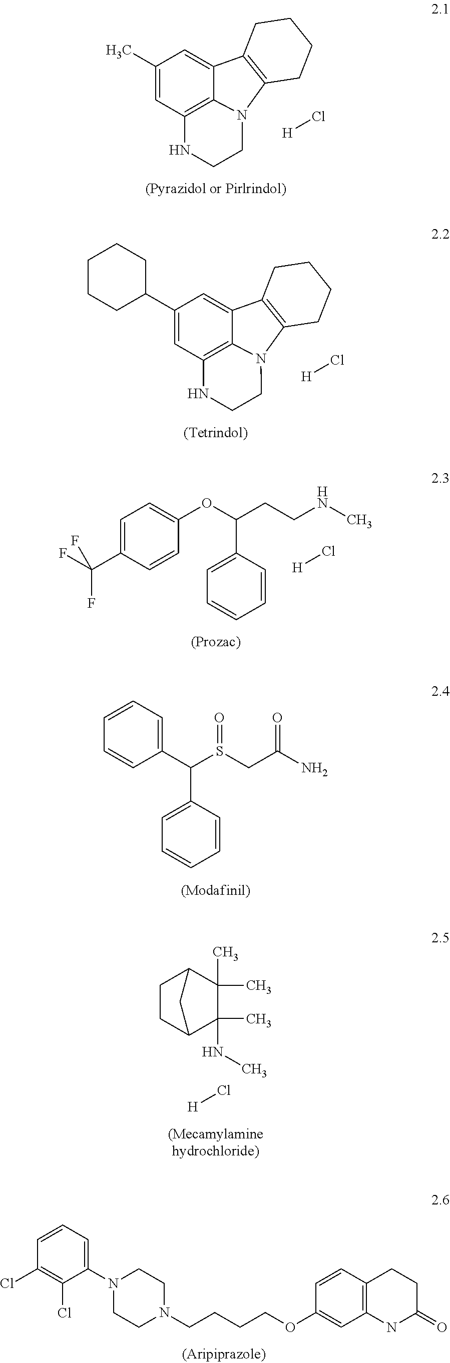

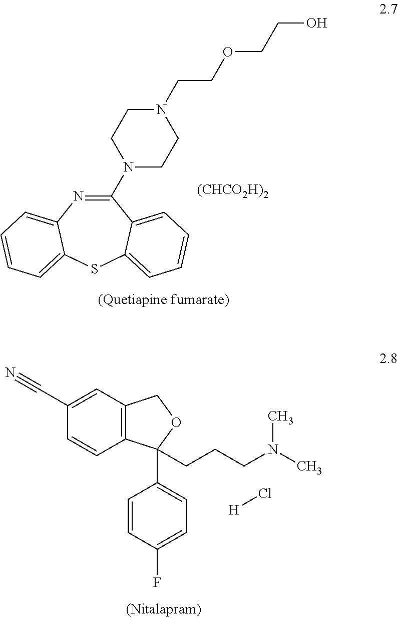

The preferred pharmaceutical composition comprises 5-methyl-2,3,7,8,9,10-hexahydro-1H-pyrazino[3,2,1-jk]carbazole hydrochloride of formula 2.1, or 5-cyclohexyl-2,3,7,8,9,10-hexahydro-1H-pyrazino[3,2,1-jk]carbazole hydrochloride of formula 2.2, or N-methyl-3-phenyl-3-(4-(trifluoromethyl)phenoxy)propane-1-amine hydrochloride of formula 2.3 as anti-depressants.

The pharmaceutical composition may include pharmaceutically acceptable excipients. Pharmaceutically acceptable excipients mean diluents, auxiliary agents and/or carriers applied in the sphere of pharmaceutics. The pharmaceutical composition in addition to the drug substance disclosed in the invention may include other active ingredients provided that they do not give rise to undesirable effects, for example, allergic reactions.

If needed, according to the present invention pharmaceutical compositions can be used in clinical practice in various forms prepared by mixing the said compositions with traditional pharmaceutical carries; for example, peroral forms (such as, tablets, gelatinous capsules, pills, solutions or suspensions); forms for injections (such as, solutions or suspensions for injections, or a dry powder for injections which requires only addition of water for injections before utilization); local forms (such as, ointments or solutions).

According to the present invention the carriers used in pharmaceutical compositions represent carriers which are used in the sphere of pharmaceutics for preparation of commonly applied forms. Binding agents, greasing agents, disintegrators, solvents, diluents, stabilizers, suspending agents, colorless agents, taste flavors are used for peroral forms; antiseptic agents, solubilizers, stabilizers are used in forms for injections; base materials, diluents, greasing agents, antiseptic agents are used in local forms.

Another embodiment of the present invention is also a method for the preparation of novel pharmaceutical composition by mixing of an effective dosage of the drug substance representing at least one substituted 1H-benzimidazole of the general formula 1 or pharmaceutically acceptable salt and/or hydrate with inert exicipient and/or solvent.

Yet another embodiment of the present invention is also a method for the preparation of novel pharmaceutical composition by mixing of an effective dosage of an anti-depressant and the drug substance representing at least one substituted 1H-benzimidazole of the general formula 1 or pharmaceutically acceptable salts and/or hydrates thereof with inert exicipient and/or solvent.

Another embodiment of the present invention is also a medicament in the form of tablets, capsules, or injections, placed in pharmaceutically acceptable packing for treatment of alcohol dependence comprising an effective dosage of the drug substance representing at least one substituted 1H-benzimidazole of the general formula 1 or pharmaceutically acceptable salts and/or hydrates thereof, or pharmaceutical composition according to the present invention.

The subject of the present invention is also a medicament in the form of tablets, capsules, or injections, placed in pharmaceutically acceptable packing for treatment of alcohol dependence comprising an effective dosage of an anti-depressant and the drug substance representing at least one substituted 1H-benzimidazole of the general formula 1 or pharmaceutically acceptable salts and/or hydrates thereof, or pharmaceutical composition according to the present invention.

The preferable medicament is the medicament comprising as the drug substance—2-ethylsulfanyl-1H-benzimidazole hydrobromide of formula 1.1 or 2-[2-(morpholin-4-yl)-ethylsulfanyl]-5-ethoxy-1H-benzimidazole dihydrochloride of formula 1.2, and as anti-depressant—5-methyl-2,3,7,8,9,10-hexahydro-1H-pyrazino[3,2,1-jk]carbazole hydrochloride of formula 2.1, or 5-cyclohexyl-2,3,7,8,9,10-hexahydro-1H-pyrazino[3,2,1-jk]carbazole hydrochloride of formula 2.2, or N-methyl-3-phenyl-3-(4-(trifluoromethyl)phenoxy)propane-1-amine hydrochloride of formula 2.3.

The more preferable medicament is the medicament in the form of tablets, capsules, or injections, placed in pharmaceutically acceptable packing for treatment of alcohol dependence representing a pharmaceutically effective combination of two active ingredients—anti-depressant and medicament comprising an effective dosage of the drug substance representing at least one substituted 1H-benzimidazole of the general formula 1 or pharmaceutically acceptable salt and/or hydrate thereof, or 2-ethylsulfanyl-1H-benzimidazole hydrobromide of formula 1.1 or 2-[2-(morpholin-4-yl)-ethylsulfanyl]-5-ethoxy-1H-benzimidazole dihydrochloride of formula 1.2, or pharmaceutical composition according to the present invention.

According to the present invention a method for treatment of alcohol dependence at humans consists in introduction of an effective dosage of the drug substance or pharmaceutical composition or an effective dosage of novel medicament.

According to the present invention the drug substance, pharmaceutical composition and medicament are employed in combination therapy of alcohol dependence at humans.

Medicaments could be introduced peroral or parenterally (for example, intravenously, subcutaneously, intraperitoneally or locally). Clinical doses of the drug substance, pharmaceutical composition or medicament comprising an effective dosage of the drug substance representing at least one substituted 1H-benzimidazole of the general formula 1 or pharmaceutically acceptable salt and/or hydrate thereof, may be corrected depending on: therapeutic efficiency and bio-accessibility of the active ingredients in patients' organism, rate of their exchange and removal from organism, and age, gender, and severity of patient's symptoms. Thus, the daily intake for adults normally being 10˜500 mg, preferably 50˜300 mg. Accordingly, the above effective doses are to be taken into consideration while preparing pharmaceutical compositions as dose units, each dose unit should contain 10˜500 mg, preferably—50˜300 mg of the drug substance. Following the instructions of physician or pharmacist, the medicaments may be taken several times over specified periods of time (preferably, from one to six times).

### BEST EMBODIMENT OF THE INVENTION

The invention is illustrated by the following FIGURES.

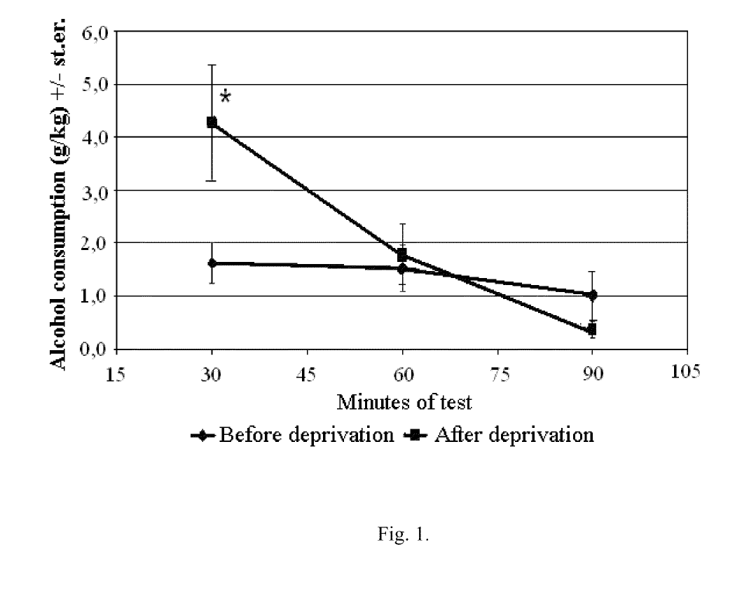

FIG. 1 Influence of 4-day's deprivation of access to alcohol solution (10%) on voluntary consumption of it by male mice of SHK line. Y-direction—consumed amount of alcohol in pure alcohol equivalent. *—alcohol-deprivation effect (ADE), Fisher LSD (ANOVA)).

Below the invention is described by means of specific examples, which illustrate but not limit the scope of the invention.

Alcohol dependence is determined by the increasing of alcohol consumption induced by short-time deprivation of alcohol. The occurrence and the level of this increasing is a criterion for self-control loss during the period of heavy drinking [Maisky A. I., Salimov R. M. Procedural guidelines for investigation of anti-alcohol medications. The guidebook for experimental (preclinical) investigation of novel pharmacological substances. Ed. Harbiev R. U., publ. “Medicine”, Moscow, 2005, p. 342-356].

For overcoming of the possible first-use gustatory rejection of alcohol solution during the first 6 days of the experiment male mice of SHK line were not given water, they had access only to alcohol solution of increasing concentration (3%—on the first and second days, 6%—on the third and fourth days and 10%—on the fifth and sixth days) [Mc Kinzie et al., Alcohol Clin Exp Res. 1998, 22(7):1584-90]. At that, free access to food was provided. During the next 2 months the animals had free access to pure water, food and 10% solution of alcohol.

For quantitative estimation of alcohol motivation the amount of voluntary alcohol (10% solution) consumption during the 90-minute's test after 4-day's alcohol deprivation was compared with the amount of alcohol consumed during the test carried out before 4-day's alcohol deprivation (alcohol-deprivation effect, ADE). ADE index was calculated as a ratio of alcohol consumption after its withdrawal (grams of pure alcohol per kilogram of body mass) to the total alcohol consumption before and after its withdrawal. The existence of alcohol dependence is determined by the value of ADE index exceeding 0.5, a lower meaning of ADE index corresponds to the absence of alcohol dependence.

At the end of 2-month's period of free access to water and alcohol the above estimation of ADE was carried out, and for further experiments the animals with ADE index exceeding 0.5 were selected. These mice, which had practically identical ADE values, were divided into groups of 9-12 animals.

Sodium γ-hydroxybutyrate was used as a reference drug in the experiments with male mice of SHK line.

### Example 1

This Example shows the reaction of mice to which during the period of deprivation sterile water was introduced in esophagus by means of not-traumatic tube. As can be seen from FIG. 1, alcohol dose consumed by mice after deprivation in the first 30 minutes of the test is 169% higher then the dose consumed before deprivation. In accordance with the present concepts this fact testifies the presence of alcohol motivation at these mice which has been enhanced by forced abstinence [Maisky A. I., Salimov R. M. Procedural guidelines for investigation of anti-alcoholic medications. The guidebook for experimental (pre-clinical) investigation of novel pharmacological substances. Ed. Harbiev R. U., publ. “Medicine”, Moscow, 2005, p. 342-356].

### Example 2

The Example shows the voluntary alcohol consumption and alcohol-deprivation effect (Table 3) at mice with formed alcohol dependence to which during the period of deprivation the following substances: placebo (sterile water), sodium γ-hydroxybutyrate, substituted 1H-benzimidazole of the general formula 1 or pharmaceutically acceptable salt and/or hydrate thereof, for example, Bemithyl 1.1 (0.5-20 mg/kg), and anti-depressant, for example, Tetrindol (2 or 10 mg/kg) were introduced in esophagus by means of (with) not-traumatic tube. Peroral administration once a day for 4 days running

TABLE 3
Voluntary alcohol consumption and alcohol-deprivation
effect at mice with formed alcohol dependence.

| | | Alcohol consumption, g/kg | | |
|---|---|---|---|---|
| | | before | after | |
| Substance | Dose, mg/kg | deprivation | | ADE |
| [symbol] | | 1.4 ± 0.65 | 4.26 ± 0.95 | 0.69 ± 0.06 |
| SHB | 150 | 2.36 ± 0.75 | 1.49 ± 1.10 | 0.45 ± 0.07 |
| 1.1 Bemithyl | 0.5 | 1.96 ± 0.75 | 4.36 ± 1.10 | 0.65 ± 0.07 |
| 1.1 | 3.0 | 3.49 ± 0.75 | 2.60 ± 1.10 | 0.46 ± 0.07 |
| 1.1 | 10.0 | 2.79 ± 0.75 | 1.59 ± 1.10 | 0.39 ± 0.07 |
| 1.1 | 20.0 | 2.56 ± 0.75 | 1.55 ± 1.10 | 0.31 ± 0.07 |
| 2.2 Tetrindol | 2.0 | 2.61 ± 0.79 | 3.42 ± 1.16 | 0.58 ± 0.08 |
| 2.2 | 10.0 | 2.61 ± 0.75 | 5.05 ± 1.10 | 0.61 ± 0.07 |
| 1.1 + 2.2 | 0.5 + 2.0 | 1.50 ± 0.71 | 0.77 ± 1.04 | 0.35 ± 0.07 |

The results, presented in Table 3, show that alcohol-deprivation effect defined as an increasing of alcohol consumption after 4-days' deprivation, is observed only for the groups of mice to which Placebo, Bemithyl 1.1 in 0.5 mg/kg dose and Tetrindol 2.2 in 2 and 10 mg/kg doses were introduced. For the rest groups of mice after introduction of Bemithyl 1.1 (3-20 mg/kg) or Bemithyl 1.1 (0.5 mg/kg) together with Tetrindol 1.2 (2 mg/kg) post-deprivation increasing of alcohol consumption was not observed. These differences are particularly demonstrated by variation in ADE index value (Table 3).

Thus, as can be seen from Table 3, the reference drug sodium γ-hydroxybutyrate (SHB) causes significant decreasing of alcohol-deprivation effect, which testifies lessening of alcohol dependence. Analogous effect is produced by substituted 1H-benzimidazoles of the general formula 1 or pharmaceutically acceptable salts and/or hydrates thereof, for example, Bemithyl 1.1 in a dose-dependent manner, the effect of which is appeared at 3 mg/kg dose and becomes more noticeable at 20 mg/kg dose. Anti-depressants, for example, Tetrindol 2.2, does not influence ADE being used alone in doses of 2-10 mg/kg. However, if maximal non-operational (sub-effective) dose of Bemithyl 1.1 (0.5 mg/kg) in combination with Tetrindol 2.2 was used, pronounced synergism of action and decreasing in ADE value to 0.35±0.07 value were observed (Table 3). These results testify that substituted 1H-benzimidazoles of the general formula 1 or pharmaceutically acceptable salts thereof (for example, [symbol] 1.1) in doses of 3-20 mg/kg or substituted 1H-benzimidazoles of the general formula 1 or pharmaceutically acceptable salts thereof in sub-effective dose together with anti-depressants (for example, Tetrindol 2.2) effectively decrease alcohol dependence.

The data shown testify the ability of novel drug substance, pharmaceutical composition and medicament comprising this drug substance to decrease the objectively registered symptoms of alcoholic dependence—increasing the consumed dose of alcohol after the period of deprivation. Besides, in comparison with the known medicaments Acamprosate or Naltrexone, novel drug substance, pharmaceutical composition and medicament comprising this drug substance, do not act unfavorably on liver function.

### INDUSTRIAL APPLICABILITY

The invention could be used in medicine, veterinary, biochemistry.

1-12. (canceled)

13. A drug substance lessening of alcohol craving representing substituted 1H-benzimidazole of the general formula 1 and pharmaceutically acceptable salt and/or hydrate thereof, 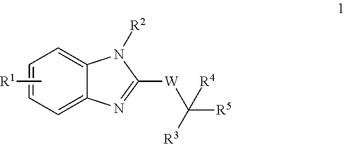
wherein:
W is S or S═O group;
R¹ is one or more substituents selected from hydrogen, halogen, C₁-C₄ alkyl, C₁-C₄ alkyloxy, optionally substituted 5-6 membered heterocyclyl with 1-2 nitrogen atoms;
R² is hydrogen or optionally substituted C₁-C₄ alkyl;
R³ and R⁴ independently of each other represent optionally identical substituents selected from hydrogen or optionally substituted C₁-C₄ alkyl;
R⁵ is an alkyl substituent selected from hydrogen, optionally substituted C₁-C₇ alkyl, C₁, C₇ alkenyl, C₂ alkynyl, optionally substituted phenyl, optionally substituted 5-6 membered heterocyclyl with 1-3 heteroatoms selected from nitrogen, oxygen and sulphur, possibly condensed with benzene ring; C₁-C₄ alkoxycarbonyl, optionally substituted aminocarbonyl;

14. The drug substance of claim 13, selected from 2-ethylsulfanyl-1H-benzimidazole hydrobromide of formula 1.1 or 2-[2-(morpholin-4-yl)-ethylsulfanyl]-5-ethyloxy-1H-benzimidazole dihydrochloride of formula 1.2.

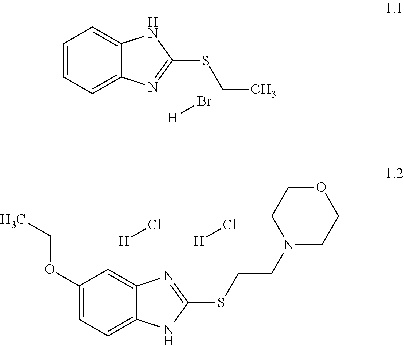

15. A pharmaceutical composition lessening of alcohol craving comprising the drug substance of the general formula 1 or pharmaceutically acceptable salts and/or hydrates thereof according to claim 13 in an effective dosage and pharmaceutically acceptable carriers, including inert excipients and/or solvents.

16. The pharmaceutical composition of claim 15 in the form of tablets, capsules, or injections placed in pharmaceutically acceptable packing.

17. The pharmaceutical composition of claim 15, comprising the drug substance selected from 2-ethylsulfanyl-1H-benzimidazole hydrobromide of formula 1.1 or 2-[2-(morpholin-4-yl)-ethylsulfanyl]-5-ethoxy-1H-benzimidazole dihydrochloride of formula 1.2 in an effective dosage.

18. A pharmaceutical composition lessening of alcohol craving comprising the drug substance of the general formula 1 or pharmaceutically acceptable salts and/or hydrates thereof according to claim 13 and anti-depressant in an effective dosage.

19. The pharmaceutical composition of claim 18 comprising the drug substance selected from 2-ethylsulfanyl-1H-benzimidazole hydrobromide of formula 1.1 or 2-[2-(morpholin-4-yl)-ethylsulfanyl]-5-ethoxy-1H-benzimidazole dihydrochloride of formula 1.2.

20. The pharmaceutical composition of claim 18 wherein said anti-depressant selected from the group representing 5-methyl-2,3,7,8,9,10-hexahydro-1H-pyrazino[3,2,1-jk]carbazole hydrochloride of formula 2.1 or 5-cyclohexyl-2,3,7,8,9,10-hexahydro-1H-pyrazino[3,2,1-jk]carbazole hydrochloride of formula 2.2, or N-methyl-3-phenyl-3-(4-(trifluoromethyl)phenoxy)propane-1-amine hydrochloride of formula 2.3.

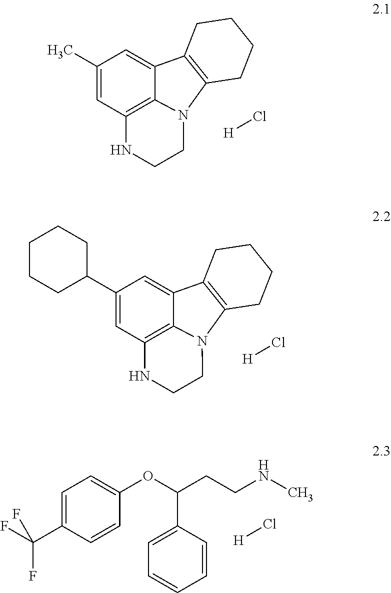

21. A method for lessening of alcohol craving comprising administering to human an effective dosage of the drug substance of the general formula 1 according to claim 13.
